(19) United States
(12) Patent Application Publication
(10) Pub. No.: US 2024/0269454 A1
(43) Pub. Date: Aug. 15, 2024

(54) Disinfecting Cap for Male and Female Needleless Connectors with Clamping Arms

(71) Applicant: Becton, Dickinson and Company, Franklin Lakes, NJ (US)

(72) Inventors: Srimannarayana Myla, Pune (IN); Rahul Malviya, Salmon Arm (CA); Atharva Shetye, Kolhapur (IN); Radhika Dharmadhikari, Pune (IN); Divik Bhargava, Meerut (IN); KVNS Jaya Chandana, Visakhapatnam (IN)

(21) Appl. No.: 18/110,019

(22) Filed: Feb. 15, 2023

(51) Int. Cl. A61M 39/16; A61M 39/20

(52) U.S. Cl. CPC A61M 39/162; A61M 39/20; A61M 2207/10

(57) ABSTRACT

A cap includes a housing having a first portion hingedly connected to a second portion. The first portion and the second portion of the housing include first ends that are connected together forming a hinge; separated second ends that define an opening of the housing; and arcuate walls extending between the first end and the second end. The cap also includes an absorbent member containing a cleaning solution, a first arm connected to the first portion of the housing, and a second arm connected to the second portion of the housing. The arms are configured such that applying a pressing force against the first arm and/or the second arm causes the first portion of the housing to pivot away from the second portion of the housing, thereby increasing an area of the opening defined by the second ends of the portions of the housing.

### BACKGROUND OF THE INVENTION

### Field of the Invention

The present disclosure relates generally to caps for medical connectors and, in particular, to a medical cap configured to be attached to either a male connector or a female connector for sealing, cleaning, and disinfecting portions of the connector.

### Description of Related Art

Vascular access devices (VADs) are commonly used medical devices, which can include intravenous (IV) catheters, such as peripheral catheters or central venous catheters. If not properly maintained or if exposed to a non-sterile environment, the VADs can become contaminated, sealed with blood clots, and/or can spread infection. Further, bacteria and other microorganisms may enter into a patient's vascular system from access hubs, ports, or valves upon connection to the VAD to deliver a fluid or pharmaceutical to a patient. Therefore, each access hub, port, valve, or other connection configured for attachment to a VAD is associated with some risk of transmitting a catheter related bloodstream infection (CRBSI) to a patient.

Many medical facilities implement sterile practices and protocols to ensure that VADs and access hubs or ports are used properly and do not become sealed or infected. These protocols often include sterilizing the access hubs, ports, and VADs, as well as flushing the catheter with a flush solution prior to use. Specifically, VAD standards of practice usually recommend flush procedures be performed after catheter placement, before fluid infusion, and before and after drug administration, blood sampling, transfusions, and/or administration of parenteral nutrition. Standards of practice can also require that access hubs, ports, and valves be capped with disinfection caps when not in use, to prevent microbial ingress into the hub, port, or valve and to sterilize areas of the hub, port, or valve that contact the VAD. Disinfection caps are disposable cap devices that contain an amount of cleaning or disinfecting solution for sterilizing portions of the port, hub, and valve.

Access hubs and ports can have a variety of different types of male or female connectors for securing the hub or port to the VAD. Currently, practitioners often carry several types of caps with them so that they can cap different types of hubs and ports, which may all be used for a particular patient. For example, caps for male needleless connectors and female needleless connectors, as well as IV and hemodialysis lines, often use different connector designs and may require different caps. There can be “male disinfecting cap devices” for disinfecting ISO594-2 type of female threaded fluid luer connectors and “female disinfecting cap devices” for disinfecting ISO594-2 type of male threaded fluid luer connectors.

Some examples of universal caps that fit on both male and female connectors are known. For example, U.S. Pat. No. 10,871,246, entitled “Universal Connector or Cap for Male and Female Threaded Fittings,” which is incorporated herein by reference in its entirety, discloses a cap including a threaded protrusion that can engage both a male connector and a female connector. However, there is a need for simpler cap designs that can be manufactured inexpensively and efficiently and which can be used with connectors having a variety of thread patterns, dimensions, and arrangements. The universal caps of the present disclosure are configured to attach to both male and female medical connectors in a secure manner sufficient for preventing microbial ingress. Further, in some examples, the universal caps of the present disclosure can be manufactured in a single-molding process.

### SUMMARY OF THE INVENTION

According to an aspect of the present disclosure, a cap configured to engage a needleless connector includes a housing having a first portion hingedly connected to a second portion. The first portion and the second portion of the housing include first ends that are connected together forming a hinge; separated second ends that define an opening of the housing; and arcuate walls extending between the first end and the second end of the first and second portions of the housing. The cap also includes at least one absorbent member disposed in the housing configured to contain a cleaning solution for cleaning and/or disinfecting portions of the needleless connector engaged to the cap. The cap also includes a first arm connected to the first portion of the housing and a second arm connected to the second portion of the housing. The arms are configured such that applying a pressing force against the first arm and/or the second arm causes the first portion of the housing to pivot away from the second portion of the housing, thereby increasing an area of the opening defined by the second ends of the first portion and the second portion of the housing.

According to another aspect of the present disclosure, a method for attaching the previously-described cap, to the needleless connector includes a step of pressing the first arm and/or the second arm radially inward to move the housing to the open position. The method also includes inserting a distal end of the needleless connector through the opening of the housing, with the housing in the open position and releasing the first arm and/or the second arm, thereby causing the housing to move to the closed position, in which the at least one absorbent member forms an interference and/or friction engagement with the needleless connector.

In accordance with an embodiment of the present invention, a cap is configured to engage a needleless connector, the cap including a housing comprising a first portion hingedly connected to a second portion, wherein the first portion and the second portion of the housing comprise first ends that are connected together forming a hinge; separated second ends that define an opening of the housing; and arcuate walls extending between the first end and the second end of the first and second portions of the housing; at least one absorbent member disposed in the housing configured to contain a cleaning solution for cleaning and/or disinfecting portions of the needleless connector engaged to the cap; a first arm connected to the first portion of the housing; and a second arm connected to the second portion of the housing, wherein the arms are configured such that applying a pressing force against the first arm and/or the second arm causes the first portion of the housing to pivot away from the second portion of the housing thereby increasing an area of the opening defined by the second ends of the first portion and the second portion of the housing.

In accordance with an embodiment of the present invention, a needleless connector includes a male luer connector or a female luer connector.

In accordance with an embodiment of the present invention, the cap is sized to receive female connectors having an outer diameter of from about 8.0 mm to about 14.0 mm and threads with a width at a crest of from about 0.3 mm to about 1.0 mm and a width at a root of the crest of from about 0.5 mm to about 1.2 mm.

In accordance with an embodiment of the present invention, the female luer connector includes a tubular body defining a tapered cavity; a septum covering an opening of the tubular body; and an external thread extending radially outward from an outer surface of the tubular body, wherein, when the female luer connector is inserted in the cap, the at least one absorbent member is configured to contact the outer surface, external thread, and septum of the female luer connector.

In accordance with an embodiment of the present invention, when the female luer connector is inserted into the cap, rotating the cap about the female luer connector causes the at least one absorbent member to scrub portions of the outer surface, external thread, and septum of the female luer connector.

In accordance with an embodiment of the present invention, the housing is configured to move between a closed position when no force is applied to the first arm and/or the second arm and an open position where force is applied to the first arm and/or the second arm, such that an area of the opening of the housing in the closed position is less than an area of the opening when the housing is in the open position.

In accordance with an embodiment of the present invention, when the housing is in the closed position, the housing forms an interference and/or friction engagement with the needleless connector which secures the cap to the needleless connector.

In accordance with an embodiment of the present invention, moving the housing to the open position removes the friction engagement between the housing and the needleless connector.

In accordance with an embodiment of the present invention, the housing is biased to the closed position.

In accordance with an embodiment of the present invention, the opening of the housing is circular in the closed position and elongated in the open position.

In accordance with an embodiment of the present invention, the cap includes a biasing member applying a biasing force to the first portion and/or the second portion of the housing that biases the housing to the closed position.

In accordance with an embodiment of the present invention, the biasing member includes a resilient annular member, such as a rubber band, positioned about outer surfaces of the arcuate walls of the first portion and the second portion.

In accordance with an embodiment of the present invention, the force applied to the first arm and/or the second arm overcomes the biasing force of the biasing member to move the housing from the closed position to the open position.

In accordance with an embodiment of the present invention, the housing includes a living hinge between the first end of the first portion and the first end of the second portion.

In accordance with an embodiment of the present invention, the arcuate wall and the second end of the first portion are separated from the arcuate wall and the second end of the second portion by a slit.

In accordance with an embodiment of the present invention, the first portion and the second portion of the housing include a rigid thermoplastic polymer comprising at least one of polyester, polycarbonate, polypropylene, polyethylene, polyethylene terephthalate, or acrylonitrile butadiene styrene.

In accordance with an embodiment of the present invention, the at least one absorbent member includes a closed first end contacting the first ends of the first and/or second portion of the housing, an open second end accessible through the opening of the housing, and an annular sidewall extending therebetween.

In accordance with an embodiment of the present invention, the at least one absorbent member defines a cylindrical interior space sized to receive the needleless connector.

In accordance with an embodiment of the present invention, the at least one absorbent member is deformable, such that an opening defined by the open second end of the at least one absorbent member moves from a circular shape to an elongated shape when the pressing force is applied to the first arm and/or the second arm.

In accordance with an embodiment of the present invention, the at least one absorbent member includes a resilient lip configured to form an interference engagement with the needleless connector for securing the cap to the needleless connector creating a seal preventing the cleaning and/or disinfecting solution from passing from an interior of the housing when the cap is engaged to the needleless connector.

In accordance with an embodiment of the present invention, the at least one absorbent member includes a sponge.

In accordance with an embodiment of the present invention, the at least one absorbent member includes an open cell foam, such as a porous foam comprising a thermoplastic elastomer.

In accordance with an embodiment of the present invention, the cap further includes the cleaning solution absorbed by the at least one absorbent member.

In accordance with an embodiment of the present invention, the cleaning solution includes Isopropyl Alcohol (IPA).

In accordance with an embodiment of the present invention, the cleaning solution includes from about 0.5% to about 3.5% chlorhexidine gluconate and about 70% IPA.

In accordance with an embodiment of the present invention, the first arm and the second arm are integral with the housing.

In accordance with an embodiment of the present invention, the first portion of the housing, the second portion of the housing, the first arm, and the second arm are integral and formed as a single molded part.

In accordance with an embodiment of the present invention, the first arm and the second arm include proximal ends connected to the arcuate wall of the first portion or the second portion of the housing and free distal ends opposite the proximal end.

In accordance with an embodiment of the present invention, the proximal ends are integral with the arcuate walls of the first portion and the second portion of the housing.

In accordance with an embodiment of the present invention, the first arm and the second arm extend distally beyond the first ends of the first portion and the second portion.

In accordance with an embodiment of the present invention, the first arm and the second arm include textured grips configured to be contacted by a user for applying the pressing force to the arms.

In accordance with an embodiment of the present invention, the first arm is spaced apart from the second arm by about 180 degrees about a longitudinal axis of the housing, and wherein the pressing force applied to the first arm is applied in a first direction and the pressing force applied to the second arm is applied in a second direction which is substantially opposite the first direction.

In accordance with an embodiment of the present invention, the cap further includes a protective cover over the opening of the housing.

In accordance with an embodiment of the present invention, the protective cover is attached to the housing by heat sealing.

In accordance with an embodiment of the present invention, a method for attaching the cap to the needleless connector includes pressing the first arm and/or the second arm radially inward to move the housing to the open position; inserting a distal end of the needleless connector through the opening of the housing, with the housing in the open position; and releasing the first arm and/or the second arm, thereby causing the housing to move to the closed position, in which the at least one absorbent member forms an interference and/or friction engagement with the needleless connector.

In accordance with an embodiment of the present invention, wherein the method further includes removing a protective cover positioned over the opening of the housing from the housing prior to inserting the distal end of the needleless connector through the opening of the housing.

### BRIEF DESCRIPTION OF THE DRAWINGS

FIG. 1A is a cross-sectional view of an exemplary male connector, as is known in the prior art.

FIG. 1B is an example of a closed female connector including a septum with a slit, as is known in the prior art.

FIG. 2A is a perspective view of a cap for a needleless connector, according to an aspect of the present disclosure.

FIG. 2B is a perspective view of another example of a cap for a needleless connector with a protective seal, according to an aspect of the present disclosure.

FIG. 2C is another perspective view of the cap of FIG. 2B.

FIG. 2D is another perspective view of the cap of FIG. 2B, with one of the arms biased radially inward to move the cap to an open position, according to an aspect of the present disclosure.

FIG. 3 is an exploded perspective view of the cap of FIG. 2B.

FIG. 4A is a front view showing a practitioner's hand manipulating the cap of FIG. 2B in order to attach the cap to a needleless connector, according to an aspect of the present disclosure.

FIG. 4B is a cross-sectional view of the cap and needleless connector of FIG. 4A.

FIG. 5A is a perspective view showing the cap of FIG. 4A connected to the needleless connector.

FIG. 5B is a cross-sectional view showing the cap and needleless connector of FIG. 4A connected together.

FIG. 5C is a perspective view of a cross-section of the cap of FIG. 4A showing a lip configured to engage the needleless connector, according to an aspect of the present disclosure.

### DESCRIPTION OF THE INVENTION

The following description is provided to enable those skilled in the art to make and use the described embodiments contemplated for carrying out the invention. Various modifications, equivalents, variations, and alternatives, however, will remain readily apparent to those skilled in the art. Any and all such modifications, variations, equivalents, and alternatives are intended to fall within the spirit and scope of the present invention.

For purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawing figures. However, it is to be understood that the invention may assume alternative variations and step sequences, except where expressly specified to the contrary. It is also to be understood that the specific devices and processes illustrated in the attached drawings, and described in the following specification, are simply exemplary embodiments of the invention. Hence, specific dimensions and other physical characteristics related to the embodiments disclosed herein are not to be considered as limiting.

The present disclosure is directed to a cap 10 configured to be connected to a medical connector 110, 112, such as an access hub, port, or valve for a VAD, by a medical practitioner to prevent the connector, port, or VAD from being contaminated by, for example, microbes, debris, or other contaminants. The medical practitioner can be a clinician or healthcare worker that performs fluid delivery or infusion procedures for patients. In particular, the “healthcare worker” can be a medical professional, such as a medical technician or nurse, trained to perform the medical procedure in accordance with sterile practices and protocols of a medical facility.

In some examples, the cap 10 can be configured to clean or disinfect portions of the connector 110, 112 or port, ensuring that the connector 110, 112 or port remains sterile prior to use. The cap 10 can be configured to remain in place on a connector 110, 112 or port for at least seven days, which is a maximum time of recommended use permitted by many medical facility sterile practice guidelines.

### Male and Female Medical Connectors

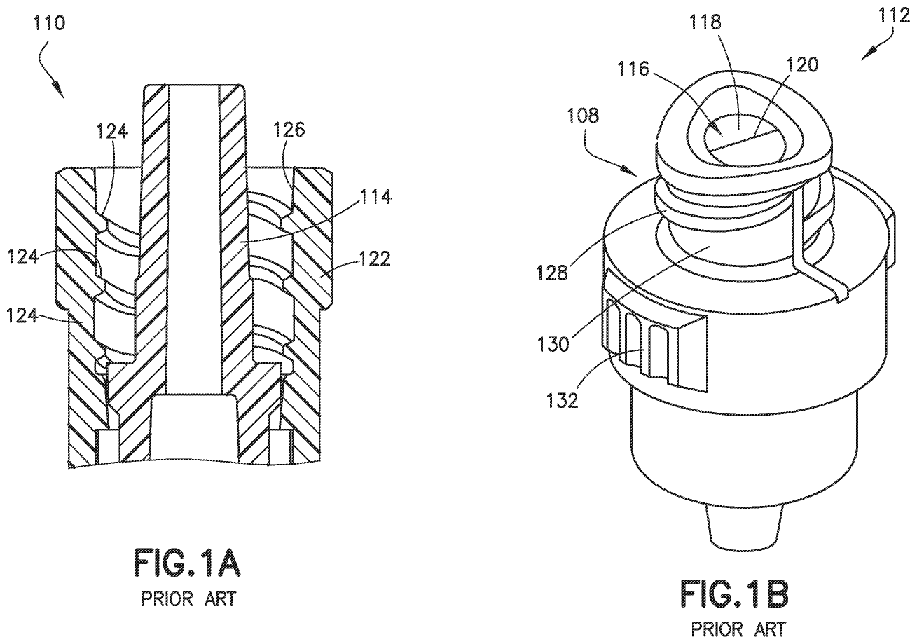

The cap 10 is a universal cap meaning that it is configured to engage with or be connected to different sizes, configurations, and/or types of medical connectors 110, 112. For example, the cap 10 can be configured to engage with or be connected to both a male connector 110 and a female connector 112. As used herein, a “male connector” refers to a connector 110 comprising an elongated member, such as a tubular member or stem 114, configured to be inserted in a tube or opening having an inner diameter that is larger than an outermost diameter of the male connector 110. An exemplary male connector 110 is shown in FIG. 1A. By contrast, a “female connector” refers to a connector 112 comprising an opening or port 116 that is configured to receive an elongated member or tubular member of another object or device in order to connect the object or device to the female connector 112. The female connector 112 can comprise an elongated distal end portion 108 with a cover or septum 118 over the opening 116. An exemplary female connector 112 including a septum 118 with a slit 120 is shown in FIG. 1B.

In some examples, the cap 10 is configured to engage different types of luer connectors 110, 112, such as both a male luer connector 110 and a female luer connector 112. For example, the cap 10 can be an appropriate size to receive a female luer connector 112 having an outer diameter of about 7.0 mm to about 10.0 mm. The cap 10 can also be sized to receive a male luer connector 110 having an outer diameter of from about 8.0 mm to about 12.0 mm.

As used herein, a “luer connector” refers to a connector 110, 112 that includes a tapered portion (e.g., a luer taper) for creating a friction engagement between a tapered stem 114 or elongated member of a male luer connector 110 and a tapered cavity. For example, the male luer connector 110 can include a tapered stem 114 or elongated member having a tapered outer surface. The female luer connector 112 can include a tapered cavity configured to receive and engage the tapered stem 114 or elongated member to connect the male luer connector 110 to the female luer connector 112.

In some examples, the male connectors 110 and the female connectors 112 can include engaging structures, such as threads, for drawing the connectors 110, 112 to another connector 110, 112 or port. For example, as shown in FIG. 1A, the male luer connector 110 can include an annular shield 122 or collar extending about the tapered stem 114 or elongated member. The annular shield 122 can include threads 124 on an inner surface 126 of the shield 122 configured to engage corresponding threads 128 on an outer surface 130 of the female luer connector 112. As shown in FIG. 1B, the female luer connector 112 includes the threads 128 extending from the outer surface 130 positioned to engage the threads 124 on the inner surface 126 of the annular shield 122 of the male luer connector 110. Twisting the female connector 112 relative to the male connector 110 causes the corresponding threads 124, 128 to engage, which draws the connectors 110, 112 together, such that the tapered stem 114 or elongated member of the male luer connector 110 moves through the opening 116 of the female connector 112. In some examples, the female connector 112 can also include vertical ribs 132 near a proximal end of the female connector 112, which can be used to manipulate the female connector 112 making it easier to twist the female connector 112 relative to another connector 110, 112 or device.

There are numerous commercially available medical devices, such as hubs, ports, and valves, which include different variations of male or female connectors 110, 112, such as male and female luer connectors. As described in further detail herein, the cap 10 of the present disclosure includes a flexible housing with a hinge, which can be opened and closed in order to engage and securely connect to different types and sizes of connectors 110, 112. For example, the cap 10 can be configured to attach to both male and female luer connectors 110, 112, such as male or female Luer-Lok™ connectors by Becton, Dickinson and Company. The cap 10 can also be configured to cover different connector designs including, without limitation, the BD Q-Syte™, BD MaxZero™, BD MaxPlus™, and SmartSite™ needle free connectors by Becton, Dickinson and Company. The cap 10 can also be configured to be connected to male and/or female connectors by other manufactures including, without limitation, MicroClave® connectors (ICU Medical Inc.) and Ultrasite® connectors (B. Braun Medical Inc.). In other examples, the cap 10 can be configured to be connect to one or more of the following commercially available male connectors: Kendall 2001NP; BD MP5303-C; ICU Med 12664-28; RyMed RYM-5307HPU; B. Braun 470108; Baxter 2C8537; Kawasumi IV-0094; Zyno B2-70071-D; B. Braun 470124; Baxter 2C7462; and Smith's Medical 536035.

### Cap for Needleless Connectors

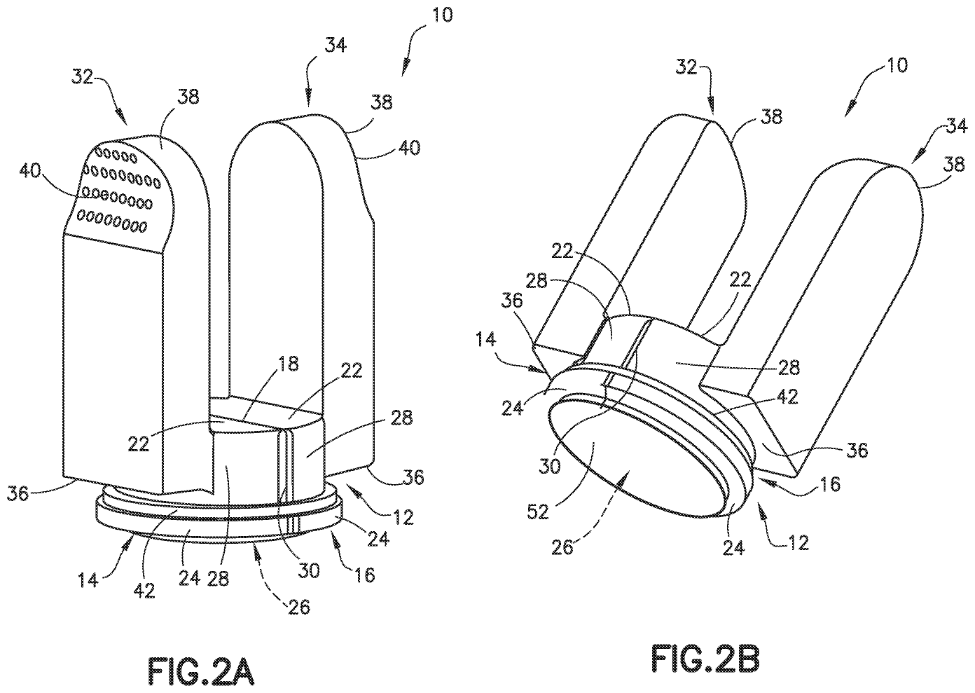

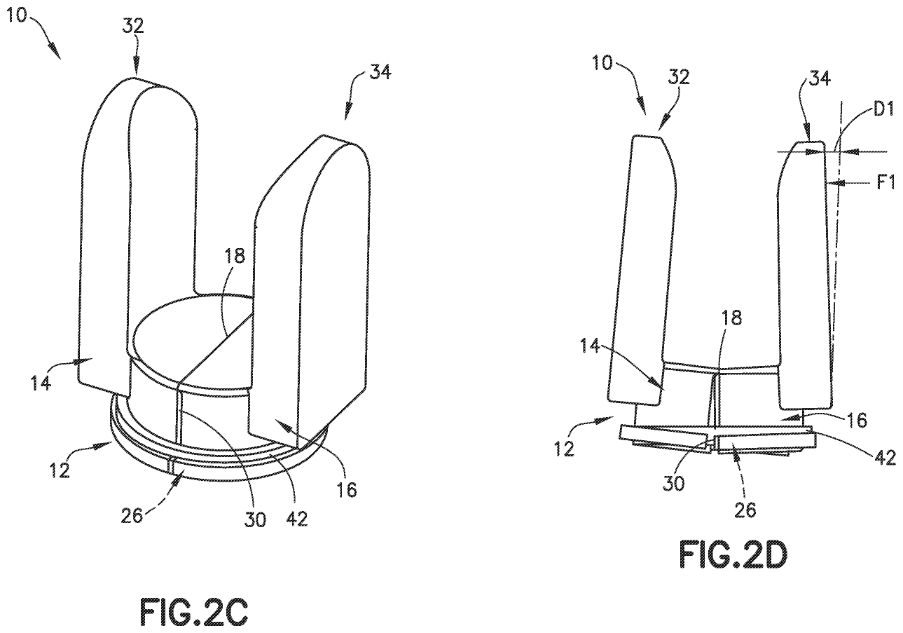

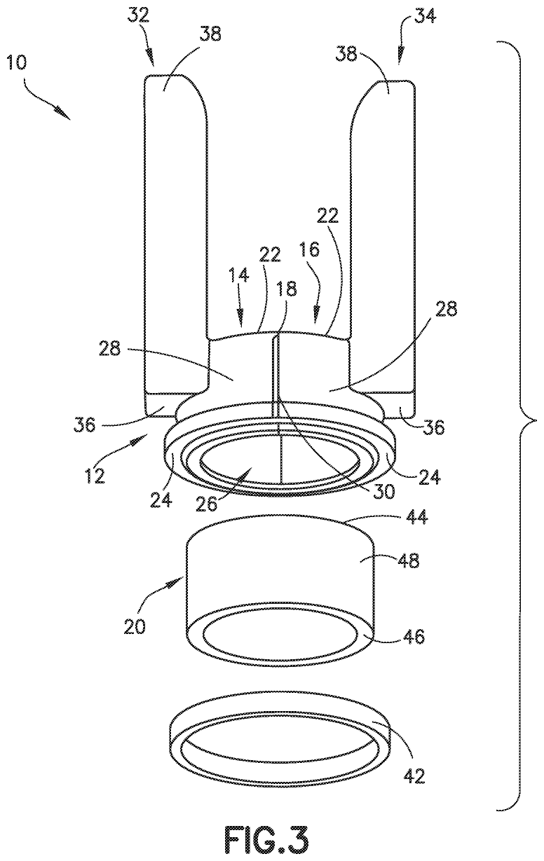

FIGS. 2A-3 illustrate an exemplary embodiment of the universal cap 10 configured to engage a needleless connector 110, 112, such as the previously described male luer connector 110 or female luer connector 112. The cap 10 comprises a housing 12 that includes a first part or portion 14 that is connected to a second part or portion 16 by a hinge 18, pivot point, or similar bendable and/or deformable structural arrangement. As described in further detail herein, the hinge 18 is configured to move from a closed or narrowest position to an expanded or open position so that the hinged housing 12 can engage or accommodate connectors 110, 112 of different configurations, shapes, and sizes. Further, because the cap 10 clamps around the connectors 110, 112 and does not directly engage threads 124, 128 of the connectors 110, 112, the cap 10 can be used with connectors 110, 112 having a variety of thread patterns, sizes, and shapes.

In some examples, the housing 12 of the cap 10, including the first portion 14 and the second portion 16, can be formed from a rigid thermoplastic polymer material, such as polyester, polycarbonate, polypropylene, polyethylene, polyethylene terephthalate, or acrylonitrile butadiene styrene. Further, the first portion 14 and the second portion 16 of the housing 12 can be integral parts formed, for example, by a single injection molding process. In other examples, the first portion 14 and the second portion 16 can be separate parts (e.g., separate parts formed by different molding processes) connected together at the hinge 18, pivot point, or joint so that the portions 14, 16 can move together to engage the needleless connector 110, 112.

As described in further detail herein, the cap 10 further comprises one or more absorbent members 20 disposed in the housing 12 configured to contain a cleaning solution for cleaning and/or disinfecting portions of the needleless connector 110, 112 engaged to the cap 10. The one or more absorbent members 20 comprise or contain cleaning and/or disinfecting solution that, when released from the absorbent member 20, contacts surfaces of the connectors 110, 112 enclosed by the cap 10 for cleaning and disinfecting the surfaces of the connectors 110, 112. Also, the absorbent members 20 can be formed from abrasive and/or porous materials that contact surfaces of the connectors 110, 112 for physically scrubbing or removing particles, dirt, dust, microbes, and other debris from the surfaces of the connectors 110, 112.

In some examples, the first portion 14 and the second portion 16 of the housing 12 include first or top ends 22 that are connected together forming the hinge 18 or joint. The first and second portions 14, 16 of the housing 12 also include separated or spaced apart second or bottom ends 24 that define an opening 26 of the housing 12 and arcuate walls 28 extending between the top end 22 and the bottom end 24 of the first and second portions 14, 16 of the housing 12. For example, the bottom ends 24 and arcuate walls 28 of the first portion 14 and the second portion 16 of the housing 12 can be separated by a narrow slit 30, slot, or gap, while the top ends 22 of the first portion 14 and the second portion 16 of the housing 12 are connected together forming the hinge 18.

In some examples, the hinge 18 between the top end 22 of the first portion 14 and the top end 22 of the second portion 16 is a living hinge 18 formed between the first portion 14 and the second portion 16 during a molding or forming process. As used herein, a “living hinge” refers to a hinge formed from a thin flexible segment or portion of an object made of a same material as two more rigid portions or segments of the object connected by the thin flexible living hinge. The living hinge 18 is capable of bending to open and close in a similar manner to hinges formed between separate rigid pieces connected to a post, pin, or rotation point.

With continued reference to FIGS. 2A-3, the cap 10 also includes elongated members, such as tabs, arms, beams, or similar structures connected to and/or extending from the housing 12 for moving the first portion 14 and the second portion 16 of the housing 12 towards or away from one another. For example, the cap 10 can include a first arm 32 connected to the first portion 14 of the housing 12 and a second arm 34 connected to the second portion 16 of the housing 12. More specifically, each of the arms 32, 34 can include bottom portions or ends 36 that are connected to and/or integral with the arcuate wall 28 of the first portion 14 or the second portion 16 of the housing 12 and free top ends 38 opposite the bottom ends 36. As shown in the figures, the arms 32, 34 extend distally beyond or above the top ends 22 of the first portion 14 and the second portion 16. As such, the top ends 38 of the arms 32, 34 can be conveniently positioned for the practitioner to manipulate using a pinching movement, such as a pinching movement between a thumb and index finger, to move the housing 12 between the closed position and the open position Further, the arms 32, 34 can be positioned on opposite sides of the housing 12, such that the first arm 32 is spaced apart from the second arm 34 by about 180 degrees about a longitudinal axis of the housing 12. In this configuration, the pressing force applied to the first arm 32 is applied in a first direction and the pressing force applied to the second arm 34 is applied in a second direction which is substantially opposite the first direction. For example, as shown in FIG. 2D, the practitioner can apply a pressing force F1 to the second arm 34, which causes the arm 34 to deflect radially inward by a distance D1.

As shown in the figures, the arms 32, 34 are positioned to permit the practitioner to apply the pressing force F1 against the first arm 32 and/or the second arm 34. Pressing against the first arm 32 and/or the second arm 34 causes the first portion 14 of the housing 12 to pivot away from the second portion 16 of the housing 12 thereby increasing an area of the opening 26 defined by the bottom ends 24 of the first portion 14 and the second portion 16 of the housing 12. Specifically, the applied pressing force F1 causes the housing 12 to transition or move between an initial or closed position (shown in FIGS. 2A-2C) and a second, expanded, or open position (shown in FIG. 2D). More specifically, the first and second portions 14, 16 of the housing 12 are configured such that an area of the opening 26 of the housing 12 in the closed position is less than an area of the opening 26 when the housing 12 is in the open position. Further, the opening 26 can be a circular shape in the closed position and an elongated, oval, or elliptical shape in the open position.

In some examples, the arms 32, 34 can include surfaces, depressions, or regions configured to be contacted or pressed by the practitioner for moving the portions 14, 16 of the housing 12 towards and away from each other. For example, as shown in FIG. 2A, the arms 32, 34 include a textured area 40 forming a grip configured to be contacted by, for example, a thumb 134 or index finger 136 of the practitioner for manipulating the arms 32, 34.

The first and second portions 14, 16 of the housing 12 are configured to engage and clamp around the portions of the needleless connectors 110, 112, thereby securing the cap 10 to the needleless connectors 110, 112. In particular, the portions 14, 16 of the housing 12 can be configured such that, when the housing 12 is in the closed position, the housing 12 forms an interference and/or friction engagement with the needleless connectors 110, 112, which secures the cap 10 to the needleless connectors 110, 112. Pressing against one or both of the arms 32, 34 effectively moves the housing 12 to the open position (shown in FIG. 2D), which overcomes or removes the friction engagement between the housing 12 and the needleless connectors 110, 112 so that the connector 110, 112 can be removed from the cap 10.

In some examples, the first and second portions 14, 16 of the housing 12 are biased to the closed position, such that when no force is applied to the arms 32, 34, the portions 14, 16 of the housing 12 clamp around the needleless connector 110, 112 securing the cap 10 to the needleless connector 110, 112. In such cases, applying a sufficient pressing force against the first arm 32 and/or the second arm 34 overcomes the biasing force and moves the portions 14, 16 of the housing 12 to the open position so that the cap 10 can be inserted onto or removed from the needleless connector 110, 112. For example, the cap 10 can include a biasing member applying the biasing force to the first portion 14 and/or the second portion 16 of housing 12 that biases the housing 12 to the closed position. In some examples, the biasing member can be a resilient annular member 42, such as an elastomeric or rubber band or cord, secured around an outer surface of the first and second portions 14, 16 of the housing 12 to hold the portions 14, 16 of the housing 12 in the closed position. As previously described, the force applied to the first arm 32 and/or the second arm 34 overcomes the biasing force of the annular resilient member 42, such as the elastomeric or rubber band, to move the housing 12 from the closed position to the open position.

The cap 10 also includes the absorbent member 20 enclosed within the housing 12, which contains the cleaning solution for cleaning and/or disinfecting portions of the needleless connectors 110, 112. For example, the absorbent member 20 can be configured to clean and disinfect surfaces of the stem 114, as well as the inner surface 126 and threads 124 of the annular shield 122 of the male connector 110. The absorbent member 20 can also be configured to clean and disinfect portions of the distal end portion 108, opening or port 116, and septum 118 of the female connector 112.

In some examples, as shown in FIGS. 3, 4B, and 5B, the absorbent member 20 is a tubular body having a closed first or top end 44 contacting the top ends 22 of the first and/or second portions 14, 16 of the housing 12, an open second or bottom end 46 accessible through the opening 26 of the housing 12, and an annular sidewall 48 extending therebetween. The closed top end 44 and the sidewall 48 can form or define a cylindrical interior space sized to receive portions of the needleless connectors 110, 112.

The absorbent member 20 can be held or retained within the cylindrical interior space or cavity defined by the first and second portions 14, 16 of the housing 12 by friction between an inner surface of the portions 14, 16 of the housing 12 and an outer surface of the absorbent member 20. In other examples, the absorbent member 20 can be held in place in the housing 12 by a conventional adhesive or mechanical fastener.

The absorbent member 20 is generally formed from a deformable material that is capable of compressing, bending and deforming as the portions 14, 16 of the housing 12 move between the closed position and the open position. The absorbent member 20 can also compress or deform to match a shape of outer surfaces of the needleless connector 110, 112 inserted into the cavity defined by the absorbent member 20. Accordingly, the absorbent member 20 can be configured to contact many portions of the outer surfaces of the connectors 110, 112 in order to scrub or mechanically remove particles, dirt, dust, microbes, and other debris from surfaces of the connectors 110, 112

In some examples, the absorbent member 20 comprises or is formed from an absorbent material capable of absorbing the cleaning or disinfecting solution for cleaning and/or disinfecting portions of the male connector 110 and the female connector 112. Further, the absorbent member 20 can be configured to radially and/or axially compress as the distal portion of the connector 110, 112 is inserted into the housing 12. The radial and/or axial compression of the absorbent member 20 can cause the cleaning solution of the absorbent member 20 to flow away from the absorbent member 20 and to contact the threads 124, 128 and other surfaces of the connector 110, 112 for cleaning and disinfecting portions of the connector 110, 112.

In some examples, the absorbent member 20 can comprise a thermoplastic elastomer, such as polypropylene, polyethylene, or synthetic or natural rubber (e.g., isoprene). The absorbent member 20 can also comprise a porous foam (e.g., an open cell foam) or sponge capable of absorbing the cleaning or disinfecting solution, such as a foam or sponge comprising polyurethane. In other examples, the foam material can be a Plastazote® foam, which is an engineered polymer foam by Zotefoams PCL.

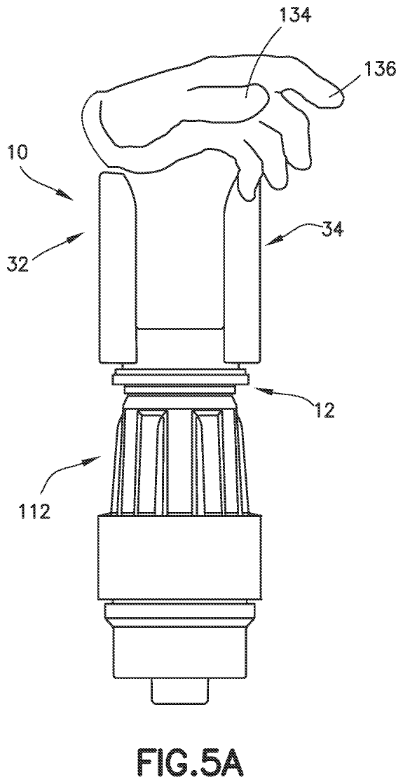

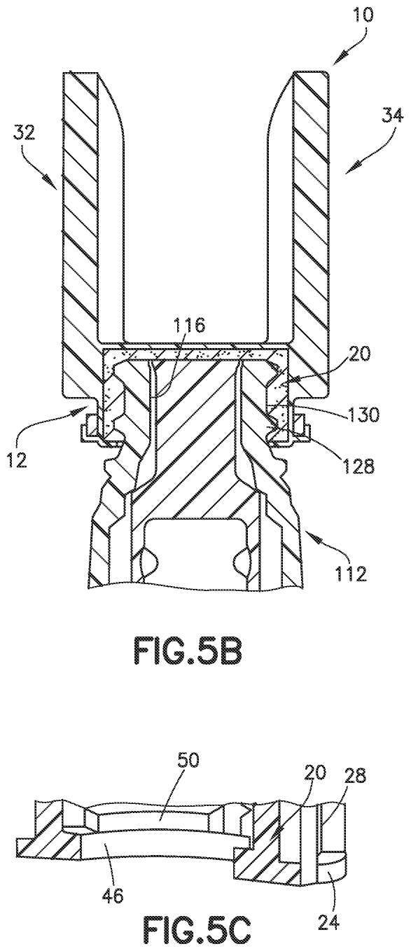

In some examples, as shown most clearly in FIG. 5C, the absorbent member 20 can also include a resilient lip 50 configured to form an interference engagement with the needleless connectors 110, 112 for securing the cap 10 to the needleless connectors 110, 112. The lip 50 can also create a seal with the outer surface of the connectors 110, 112 preventing the cleaning and/or disinfecting solution from passing or leaking away from an interior of the housing 12 when the cap 10 is engaged to the needleless connectors 110, 112.

In some examples, the absorbent member 20 is provided (e.g., presoaked) with the cleaning or disinfecting solution, such as during manufacturing of the cap 10. The cleaning or disinfecting solution can be an antimicrobial, anti-fungal, antibacterial, or antiviral solution that cleans and sterilizes surfaces of the connectors 110, 112. In some examples, the cleaning solution can be isopropyl alcohol (IPA), such as about 70% IPA. In other examples, the cleaning solution can be about 0.5% to about 3.5% chlorhexidine gluconate in combination with about 70% IPA. A chlorohexidine composition may be beneficial because it has a slower evaporation rate than IPA and, therefore, can provide a more persistent disinfectant activity after the cap 10 is removed from the connector 110, 112 and before the VAD is connected to the hub, port, or valve.

In some examples, the cap 10 can further comprise a removable and/or disposable protective cover 52 (shown in FIG. 2B) positioned over the opening 26 of the housing 12. The protective cover 52 can be provided to protect components and portions of the cap 10, such as the housing 12 and absorbent member 20, during transport and storage to prevent contamination and to prevent the cleaning or disinfecting solution from evaporating prior to use. The protective cover 52 can comprise a sheet, such as a polymer film, with adhesive on a first side of the sheet for removably mounting the protective cover 52 to the opening 26 of the housing 12. Alternatively, the protective cover 52 can be removably mounted to the open bottom 24 of the housing 12 by heat sealing. The protective cover 52 can be formed from a material that is impervious or substantially impervious to air, so that the cleaning or disinfecting solution on the absorbent member 20 does not evaporate or dry-out. Accordingly, the protective cover 52 can increase a shelf life of the cap 10, as well as prevent microbes and other debris from collecting in the cap 10 prior to use.

### Method for Attaching a Connector to the Cap

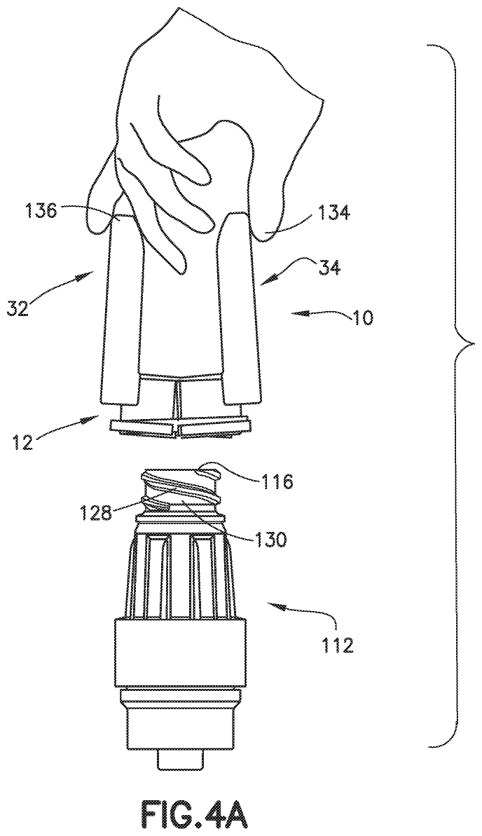

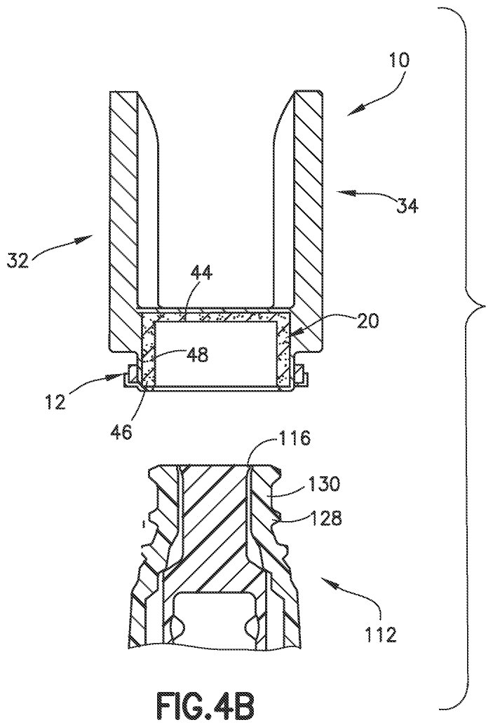

As previously described, the cap 10 of the present disclosure is a universal cap 10 configured to be connected to various types and sizes of male connectors 110 and female connectors 112. For example, FIGS. 4A and 4B show the cap 10 prior to being connected to a female connector 112. FIGS. 5A and 5B show the cap 10 connected to the female connector 112.

In order to connect the cap 10 to a female connector 112, the practitioner first removes any packaging from the cap 10 and removes the protective cover 52 from the opening 26 at the bottom end 24 of the portions 14, 16 of the housing 12. Once the packaging and protective cover 52 are removed, the practitioner applies pressure to one or both of the arms 32, 34 causing the portions 14, 16 of the housing 12 to pivot away from each other. For example, as shown in FIG. 4A, the practitioner may press the first arm 32 with an index finger 136 and press the second arm 34 with the thumb 134 causing the portions 14, 16 of the housing 12 to move from the closed positon to the open position.

With the housing 12 in the open position, the practitioner moves the cap 10 toward the female connector 112 inserting the opening 26 of the cap 10 over the opening 116 and septum 118 of the female connector 112. Continuing to move the female connector 112 into the housing 12 brings outer surfaces 130 and threads 128 of the female connector 112 into contact with the annular sidewall 48 of the absorbent member 20. The practitioner can continue to advance the female connector 112 into the cap 10 eventually causing the opening 116 and septum 118 of the female connector 112 to contact and/or press against the closed top end 44 of the absorbent member 20, as shown in FIG. 5B. As previously described, contact between abrasive surfaces of the absorbent member 20 and surfaces of the female connector 112 can mechanically remove particles, such as microbes and other debris, from surfaces of the female connector 112, which contributes to the cleaning effect provided by the cap 10. In some examples, once the female connector 112 is fully engaged to the cap 10, the practitioner can also twist or rotate the cap 10 relative to the female connector 112 in order to enhance the scrubbing effect of the absorbent member 20 against surfaces of the female connector 112 in order to remove additional particles, dirt, dust, microbes, and debris from surfaces of the female connector 112.

In some examples, the contact between the surfaces of the female connector 112 and the absorbent member 20 can radially and/or axially compress the absorbent member 20. Compression of the absorbent member 20 releases the cleaning or disinfecting solution, causing the cleaning or disinfecting solution to contact portions of the connector 112. In particular, the cleaning solution can contact and disinfect portions of the female connector 112 including the septum 118, as well as outer surfaces 130 and threads 128 of the tubular distal portion 108 of the female connector 112.

Once the cap 10 is fully engaged to the female connector 112 with sufficient pressure to scrub particles from surfaces of the female connector 112 and to release the cleaning or disinfecting solution from the absorbent member 20, as shown in FIG. 5A, the practitioner can release the arms 32, 34. Releasing the arms 32, 34 causes the portions 14, 16 of the housing 12 to cinch or clamp around the female connector 112 forming a secure engagement between the cap 10 and the female connector 112.

In order to remove the cap 10 from the female connector 112, the practitioner grasps the arms 32, 34 as previously described, and applies pressure to the first arm 32 or the second arm 34, which causes the portions 14, 16 of the housing 12 to move to the open position. With the housing 12 in the open position, the practitioner pulls the cap 10 axially away from the female connector 112, thereby releasing the female connector 112 from the cap 10. Once removed, the cap 10 can be discarded, as it is often a single use product.

Once the female connector 112 is fully removed from the housing 12, the female connector 112 can be connected to a VAD. For example, the female connector 112 can be attached or inserted into a hub, port, or valve of the VAD forming a needleless fluid-tight connection between the female connector 112 and a fluid path, channel, or lumen of the VAD.

As previously described, the universal cap 10 of the present disclosure can also be connected to a male connector 110 in order to form a secure and sterile engagement with the male connector 110, which prevents or reduces contamination of the male connector 110. For example, the cap 10, with the housing 12 in the open position, can be inserted over the male connector 110, such that the stem 114 and annular shield 122 of the male connector 110 are inserted into the housing 12 and contact portions of the absorbent member 20. In particular, the annular shield 122 can contact the annular sidewall 48 of the absorbent member 20, while the stem 114 contacts the closed top end 44 of the absorbent member 20. As previously described, contact between surfaces of the male connector 110 and the absorbent member 20 can scrub or mechanically remove particles from the surfaces of the male connector 110. The practitioner can also twist or rotate the cap 10 relative to the male connector 110 to enhance the scrubbing effect of the absorbent member 20 against surfaces of the male connector 110. The contact between the male connector 110 and the absorbent member 20 also releases the cleaning or disinfecting solution from the absorbent member 20 causing the cleaning and/or disinfecting solution to contact and disinfect surfaces of the stem 114 and annular shield 122 of the male connector 110. The cap 10 can be removed from the male connector 110 in the same manner as for the female connector 112, as previously described.

While examples of the universal cap 10 and methods of use of the present disclosure are shown in the accompanying figures and described hereinabove in detail, other examples will be apparent to, and readily made by, those skilled in the art without departing from the scope and spirit of the invention. Accordingly, the foregoing description is intended to be illustrative rather than restrictive. The invention described hereinabove is defined by the appended claims and all changes to the invention that fall within the meaning and the range of equivalency of the claims are to be embraced within their scope.

The invention claimed is:

1. A cap configured to engage a needleless connector, the cap comprising:
a housing comprising a first portion hingedly connected to a second portion, wherein the first portion and the second portion of the housing comprise first ends that are connected together forming a hinge; separated second ends that define an opening of the housing; and arcuate walls extending between the first end and the second end of the first and second portions of the housing;
at least one absorbent member disposed in the housing configured to contain a cleaning solution for cleaning and/or disinfecting portions of the needleless connector engaged to the cap;
a first arm connected to the first portion of the housing; and
a second arm connected to the second portion of the housing,
wherein the arms are configured such that applying a pressing force against the first arm and/or the second arm causes the first portion of the housing to pivot away from the second portion of the housing thereby increasing an area of the opening defined by the second ends of the first portion and the second portion of the housing.

2. The cap of claim 1, wherein the needleless connector comprises a male luer connector or a female luer connector.

3. The cap of claim 2, wherein the cap is sized to receive female luer connectors having an outer diameter of from about 8.0 mm to about 10.0 mm and threads with a width at a crest of from about 0.3 mm to about 1.0 mm and a width at a root of the crest of from about 0.5 mm to about 1.2 mm.

4. The cap of claim 2, wherein the female luer connector comprises:
a tubular body defining a tapered cavity;
a septum covering an opening of the tubular body; and
an external thread extending radially outward from an outer surface of the tubular body,
wherein, when the female luer connector is inserted in the cap, the at least one absorbent member is configured to contact the outer surface, the external thread, and the septum of the female luer connector.

5. The cap of claim 4, wherein, when the female luer connector is inserted into the cap, rotating the cap about the female luer connector causes the at least one absorbent member to scrub portions of the outer surface, the external thread, and the septum of the female luer connector.

6. The cap of claim 1, wherein the housing is configured to move between a closed position when no force is applied to the first arm and/or the second arm and an open position when force is applied to the first arm and/or the second arm, such that an area of the opening of the housing in the closed position is less than an area of the opening when the housing is in the open position.

7. The cap of claim 1, wherein, when the housing is in a closed position, the housing forms an interference and/or friction engagement with the needleless connector which secures the cap to the needleless connector.

8. The cap of claim 7, wherein moving the housing to an open position removes the friction engagement between the housing and the needleless connector.

9. The cap of claim 6, wherein the housing is biased to the closed position.

10. The cap of claim 6, further comprising a biasing member applying a biasing force to the first portion and/or the second portion of housing that biases the housing to the closed position,
wherein the biasing member comprises a resilient annular member positioned about outer surfaces of the arcuate walls of the first portion and the second portion.

11. The cap of claim 10, wherein the force applied to the first arm and/or the second arm overcomes the biasing force of the biasing member to move the housing from the closed position to the open position.

12. The cap of claim 1, wherein the housing comprises a living hinge between the first end of the first portion and the first end of the second portion.

13. The cap of claim 1, wherein the at least one absorbent member comprises a closed first end contacting the first ends of the first and/or second portion of the housing, an open second end accessible through the opening of the housing, and an annular sidewall extending therebetween.

14. The cap of claim 1, further comprising the cleaning solution absorbed by the at least one absorbent member, wherein the cleaning solution comprises Isopropyl Alcohol (IPA) and/or chlorhexidine gluconate.

15. The cap of claim 1, wherein the first portion of the housing, the second portion of the housing, the first arm and the second arm are integral and formed as a single molded part.

16. The cap of claim 1, wherein the first arm and the second arm comprise proximal ends integral with the arcuate wall of the first portion or the second portion of the housing and free distal ends opposite the proximal ends.

17. The cap of claim 16, wherein the distal ends of the first arm and the second arm are axially spaced apart from and above the first ends of the first portion and the second portion.

18. The cap of claim 1, wherein the first arm is spaced apart from the second arm by about 180 degrees about a longitudinal axis of the housing, and
wherein the pressing force applied to the first arm is applied in a first direction and the pressing force applied to the second arm is applied in a second direction which is substantially opposite the first direction.

19. The cap of claim 1, further comprising a protective cover over the opening of the housing.

20. A method for attaching the cap of claim 1 to the needleless connector, the method comprising:
pressing the first arm and/or the second arm radially inward to move the housing to an open position;
inserting a distal end of the needleless connector through the opening of the housing, with the housing is in the open position; and
releasing the first arm and/or the second arm, thereby causing the housing to move to a closed position, in which the at least one absorbent member forms an interference and/or friction engagement with the needleless connector.
